(19) United States
(12) Patent Application Publication
(10) Pub. No.: US 2019/0388117 A1
(43) Pub. Date: Dec. 26, 2019

(54) SURGICAL SHAVER WITH FEATURE TO DETECT WINDOW STATE

(71) Applicant: Acclarent, Inc., Irvine, CA (US)

(72) Inventors: Fatemeh Akbarian, Rancho Palos Verdes, CA (US); Itzhak Fang, Irvine, CA (US); Jetmir Palushi, Irvine, CA (US)

(21) Appl. No.: 16/012,922

(22) Filed: Jun. 20, 2018

(51) Int. Cl. A61B 17/3207

(52) U.S. Cl. CPC A61B 17/320758; A61B 2017/00398

(57) ABSTRACT

A surgical instrument for cutting a tissue includes a shaft, a cutting member, and an alignment system. The shaft extends along a longitudinal axis and includes a shaft opening. The cutting member is disposed within the shaft lumen to cyclically move from an open state to a closed state. The cutting member includes a sidewall, a cutting window, and a suction lumen. The cutting window opening in the open state aligns with the shaft opening such that the cutting window opening and the shaft opening are in fluid communication. The sidewall in the closed state aligns with the shaft opening such that the sidewall blocks fluid communication to the shaft opening for inhibiting suctioning the tissue therethrough. The alignment system urges movement of the cutting member moving the open state toward the closed state such that the cutting member stops in the closed state.

### BACKGROUND

Surgical cutting instruments configured for removal of lesions, polyps and fibroids within the nasal cavity are known. Some configurations may include an elongated inner member rotatably coaxially disposed within a tubular outer member. The distal end of the outer member includes an opening, and the distal end of the inner member includes cutting edges. The proximal ends of the two members may be connected to a handle directly or via a detachable hub. The inner member may be hollow and in communication with an aspiration port so that severed tissue, etc. can be aspirated out through the hollow member. The cutting edges can have any various configurations suitable for the particular type of tissue, such as bone tissue, to be done, with the opening configured to cooperate with the specific cutting edge configuration.

To use such surgical cutting instrument to address such tissues, the opening/cutting edge is advanced to the target surgical site, and the opening positioned adjacent the tissue to be removed. The opening may be repositioned to address tissue which could not be accessed with the instrument in the previous position. Surgical cutting instruments with a fixed opening allow surgeons to cut only in the direction of the fixed opening cutting. To access, cut and remove tissue at various locations, surgeons have to reposition the instrument at various angles; or in some instances, change to other instruments having a more appropriately arranged opening.

It may be desirable to access, cut and remove tissue, such as bone tissue, at various locations without having to reposition or change the surgical instrument. While several different surgical instruments and methods of use have been made for tissue removal within the nasal cavity, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of a first exemplary surgical cutting instrument having a handle assembly and a shaft assembly;

FIG. 2 depicts an exploded perspective fragmentary view of the shaft assembly of FIG. 1 having a shaft and a cutting member;

FIG. 3 depicts a perspective view of a second exemplary surgical cutting instrument having a handle assembly, a shaft assembly, and a shaft assembly alignment system;

FIG. 4 depicts a partially exploded perspective view of the surgical cutting instrument of FIG. 3;

FIG. 5 depicts a perspective view of the handle assembly of FIG. 3;

FIG. 6 depicts a right side perspective view of an outer coupling knob of the shaft assembly of FIG. 3 showing a search coil sensor;

FIG. 7 depicts a left side perspective view of the outer coupling knob of the shaft assembly of FIG. 3 showing another search coil sensor;

FIG. 8 depicts an enlarged perspective view of the surgical cutting instrument of FIG. 3 having various components hidden for improved clarity of the search coil sensor of FIG. 7;

FIG. 9 depicts an enlarged perspective view of the shaft assembly of FIG. 3 with various components hidden for improved clarity of rotation of the outer coupling knob with the search coil sensor and an outer shaft extending therefrom;

FIG. 10A depicts an enlarged perspective view of the shaft assembly of FIG. 3 with various components hidden for improved clarity of rotation of a cutting member relative to the outer shaft and the search coil sensor in a closed state;

FIG. 10B depicts the enlarged perspective view of the shaft assembly similar to FIG. 10A, but with the cutting member rotating from the closed state toward an open state;

FIG. 10C depicts the enlarged perspective view of the shaft assembly similar to FIG. 10A, but with the cutting member rotating in the open state;

FIG. 11A depicts a diagrammatic view of the shaft assembly alignment system determining that the shaft assembly is in the open state of FIG. 10C;

FIG. 11B depicts a diagrammatic view of the shaft assembly alignment system determining that the shaft assembly is in the closed state of FIG. 10A;

FIG. 12 depicts a schematic view of a detection of the open and closed states via the search coil sensors of the shaft assembly alignment system of FIGS. 11A-11B;

FIG. 13 depicts a perspective view of a third exemplary surgical cutting instrument having a handle assembly and an alternative shaft assembly alignment system with a pair of shaft encoders for use with the shaft assembly of FIG. 3 for determining that the shaft assembly is in the closed state;

FIG. 14 depicts an enlarged side sectional view of a fourth exemplary surgical cutting instrument taken along a centerline thereof having a handle assembly, a shaft assembly, and a suction alignment system with a pressure sensor for determining that the shaft assembly is in the closed state;

FIG. 15 depicts a perspective view of a fifth exemplary surgical cutting instrument having a handle assembly, a shaft assembly, and an engagement alignment system;

FIG. 16A depicts a distal end view of the shaft assembly of FIG. 15 with the cutting member rotating from the open state toward the closed state;

FIG. 16B depicts the distal end view of the shaft assembly similar to FIG. 16A, but with the cutting member in the closed state;

FIG. 17A depicts a cross-sectional view of the shaft assembly of FIG. 15 taken along section line 17A-17A with the cutting member rotating from the open state toward the closed state as shown in FIG. 16A; and

FIG. 17B depicts the cross-sectional view of the shaft assembly similar to FIG. 17A, but with the cutting member in the closed state as shown in FIG. 16B.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “left,” “right,” “side,” “axial,” and “longitudinal” also are used herein for reference to relative positions and directions. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Surgical Cutting Instrument

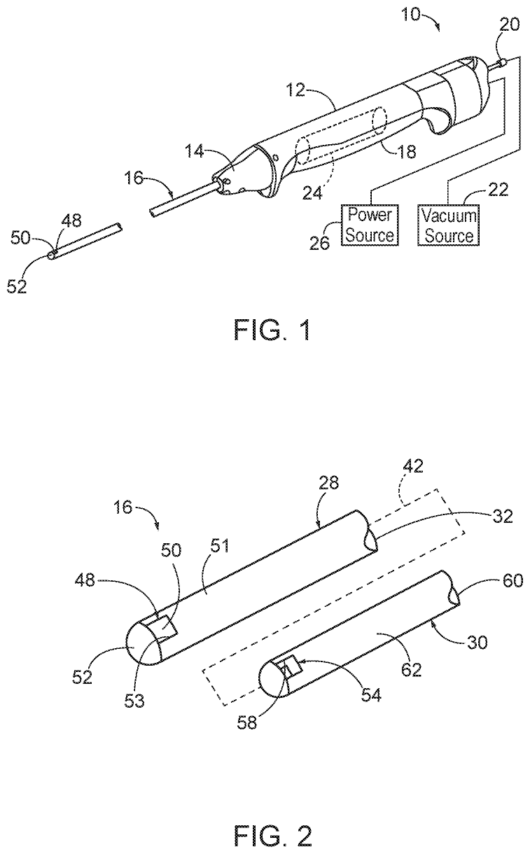

FIG. 1-2 show a first exemplary surgical cutting instrument (10) that may be used to remove tissue, such as bone tissue, from the nasal cavity, as well as from any other suitable location. Surgical cutting instrument (10) of the present example includes a handle assembly (12), a hub (14), and a first shaft assembly (16) extending distally from handle assembly (12). Handle assembly (12) has a handle (18) which may be of any suitable configuration. Handle (18) may include controls for the operation of surgical cutting instrument (10), or the controls may be located remotely. Surgical cutting instrument (10) further includes a suction port (20) operatively connected to a vacuum source (22) and configured to enable aspiration of tissue, such as a bone tissue, from a surgical site. Rotational motion is delivered by a motorized drive assembly (24) within handle assembly (12) to shaft assembly (16) in the present example, although any suitable rotational or oscillatory motion source may be utilized. For example, such motion source may be housed within handle assembly (12) or may be external and connectable to handle assembly (12). A power source (26) connects to motorized drive assembly (24) to power surgical cutting instrument (10) for use. In addition or alternatively, handle assembly (12) may house a battery (not shown).

Shaft assembly (16) generally includes an outer shaft (28) and an inner cutting member (30) collectively configured to receive and remove tissue from the surgical site. Cutting member (30), which is illustrated as a tube, is disposed within a longitudinally extending lumen (32) of shaft (28). Cutting member (30) is configured to be rotated about a longitudinal axis (42) of shaft assembly (16) at a distal portion. Although shaft assembly (16) is depicted as rigid, all or a portion of shaft assembly (16) may be flexible, with longitudinal axis (42) comprising a series of cross-sectional centers. Cutting member (30) defines a lumen and extends proximally to handle assembly (12) and connects to motorized drive assembly (24), which rotatably drives cutting member (30) relative to shaft (28). In the present example, shaft (28) is formed of polycarbonate and cutting member (30) is formed of stainless steel. Of course, shaft (28) and cutting member (30) may be formed of one or more alternative materials in accordance with the invention described herein. The invention is thus not intended to be unnecessarily limited to manufacture with polycarbonate and stainless steel. While the present example of cutting member (30) is a hollow tube, cutting member (30) is not limited to being tubular and defining its own lumen (32).

Shaft (28) includes a window region (48) having a shaft opening, such as a shaft window opening (50), at distal portion. Distal portion includes a tubular sidewall (51) that distally terminates in a curved end, such as a generally hemispherical end (52). Shaft window opening (50) extends through tubular sidewall (51) of shaft (28) into lumen (32) and is in fluid communication with the environment surrounding shaft (28). Shaft window opening (50) faces radially outward relative to longitudinal axis (42) such that tissue is configured to be radially received through shaft window opening (50) into a central suction lumen (60) of cutting member (30) in a radially inward direction. Shaft window opening (50) is surrounded by a relatively dull edge (53).

Cutting member (30) includes a cutting window opening (54) at distal portion of cutting member (30). Cutting window opening (54) is configured to longitudinally align with shaft window opening (50) and includes a cutting edge (58) extending therealong. It is noted that less than the entirety of cutting edge (58) may be configured for cutting tissue against an opposing edge (53) of shaft (28). At least a portion of cutting edge (58) is disposed to move adjacent to and across at least a portion of window region (48) when cutting member (30) is rotated or oscillated about longitudinal axis (42). By way of example, as cutting member (30) moves in a clockwise direction, edge (53) of window region (48) provides an opposing surface to cutting edge (58) whereby tissue may be severed to remove a cut tissue portion therefrom. Cutting edge (58) and edge (53) may have any configuration which suitably cooperates with the other to sever tissue, such as a knife edge, a serrated edge, bipolar, monopolar or harmonic energy modality, or laser activated cutting edge.

The extent of movement and position of cutting edge (58) relative to edge (53) is sufficient to separate tissue, whether by severing, tearing or any other mechanism. For example, cutting edge (58) may cyclically move across at least a portion of window region (48). Further clockwise movement of cutting member (30) will advance cutting edge (58) past edge (53), such as results from oscillation about longitudinal axis (42) or from full rotation about longitudinal axis (42).

With continued reference to FIGS. 1-2, vacuum source (22) generates suction in a proximal direction along suction lumen (60) and longitudinal axis (42) toward suction port (20). Suction lumen (60) is defined by a tubular sidewall (62) of cutting member (30) in the present example and is in direct fluid communication with cutting window opening (54). Without tissue blocking cutting window opening (54), such suction proximally withdraws a window airflow therethrough along suction lumen (60). However, once tissue is respectively introduced into window opening (54), suction effectively draws tissue into window opening (54) for resection while tissue blocks airflow along suction lumen (60). Airflow through suction lumen (60) essentially terminates such that vacuum source (22) accumulates the vacuum within suction lumen (60). Such termination of airflow may generally be referred to as a stalled airflow within lumen. Additional details regarding airflow through lumen and aspiration vents for improving such airflow are discussed in alternative examples described in U.S. patent application Ser. No. 15/795,473, entitled “Tissue Shaving Instrument,” filed Oct. 27, 2017, the disclosure of which is incorporated by reference herein.

Furthermore, surgical cutting instrument (10) may be used in conjunction with an image-guide surgery (IGS) navigation system, medical procedure chair, and displays described alone or in any combination according to the following: U.S. Pat. Pub. No. 2016/0008083, entitled “Guidewire Navigation for Sinuplasty,” published Jan. 14, 2016; U.S. Patent App. No. 62/555,824, entitled “Apparatus to Secure Field Generating Device to Chair,” filed Sep. 8, 2017; U.S. Pat. Pub. No. 2016/0008083, entitled “Guidewire Navigation for Sinuplasty,” published Jan. 14, 2016; U.S. Pat. Pub. No. 2016/0310042, entitled “System and Method to Map Structures of Nasal Cavity,” published Oct. 27, 2016; U.S. Pat. No. 8,702,626, entitled “Guidewires for Performing Image Guided Procedures,” issued Apr. 22, 2014; U.S. Pat. No. 8,320,711, entitled “Anatomical Modeling from a 3-D Image and a Surface Mapping,” issued Nov. 27, 2012; U.S. Pat. No. 8,190,389, entitled “Adapter for Attaching Electromagnetic Image Guidance Components to a Medical Device,” issued May 29, 2012; U.S. Pat. No. 8,123,722, entitled “Devices, Systems and Methods for Treating Disorders of the Ear, Nose and Throat,” issued Feb. 28, 2012; U.S. Pat. No. 7,720,521, entitled “Methods and Devices for Performing Procedures within the Ear, Nose, Throat and Paranasal Sinuses,” issued May 18, 2010; U.S. Pat. Pub. No. 2014/0364725, entitled “Systems and Methods for Performing Image Guided Procedures within the Ear, Nose, Throat and Paranasal Sinuses,” published Dec. 11, 2014; U.S. Pat. Pub. No. 2014/0200444, entitled “Guidewires for Performing Image Guided Procedures,” published Jul. 17, 2014; U.S. Pat. No. 9,198,736, entitled “Adapter for Attaching Electromagnetic Image Guidance Components to a Medical Device,” issued Dec. 1, 2015; U.S. Pat. Pub. No. 2011/0060214, entitled “Systems and Methods for Performing Image Guided Procedures within the Ear, Nose, Throat and Paranasal Sinuses,” published Mar. 10, 2011; U.S. Pat. No. 9,167,961, entitled “Methods and Apparatus for Treating Disorders of the Ear Nose and Throat,” issued Oct. 27, 2015; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosures of each of the these references being incorporated by reference herein.

II. Alignment System to Position Cutting Window Opening in a Closed State

While surgical cutting instrument (10) is configured to remove a target tissue as discussed above in greater detail, such surgical cutting instrument (10) may be inserted and removed alongside a variety of nearby tissues to access the target tissue. In the event that cutting window opening (54) is in an opened state, suction applied by vacuum source (22) may withdraw any such nearby tissues into surgical cutting instrument (10) and, in turn, cause the patient discomfort, pain, or even inadvertent removal of the tissue. In contrast, positioning cutting window opening (54) in a closed state prior to such insertion or removal effectively terminates suction through cutting window opening (54) in order to inhibit in inadvertently withdrawing tissue into surgical cutting instrument (10). Thus, determining the state of cutting window opening (54) and/or actively positioning cutting window opening (54) to the closed state in order to insert or remove surgical cutting instrument (10) provides for greater comfort and enhanced outcomes for the patient.

Various surgical cutting instruments (110, 210, 310, 410) discussed below incorporate an alignment system (114, 214, 314, 414) for determining the state of cutting window opening (54) and/or actively positioning cutting window opening (54) to the closed state. Three such surgical cutting instruments (110, 210, 310) with alignment systems (114, 214, 314) monitor the position of cutting member (30) relative to shaft (28) and detect the closed state of cutting window opening (54) to determine whether cutting window opening (54) is in the closed state to inhibit suction or the open state to allow suction therethrough. Cutting window opening (54) may then be actively positioned to the closed state as desired based on such determination. Another surgical cutting instrument (410) with alignment system (414) is configured to actively engage cutting member (30) such that cutting window opening (54) is positioned in the closed state without such passive detection and monitoring. Such active and passive features are not intended to be mutually exclusive, and any feature or associated use of the various alignment systems (114, 214, 314, 414) may be used alone or in combination with each other. The invention is thus not intended to be unnecessarily limited to the particular examples shown herein. In any case, like numbers provided below indicate like features discussed above in greater detail.

A. Shaft Assembly Alignment System with Shaft Sensors

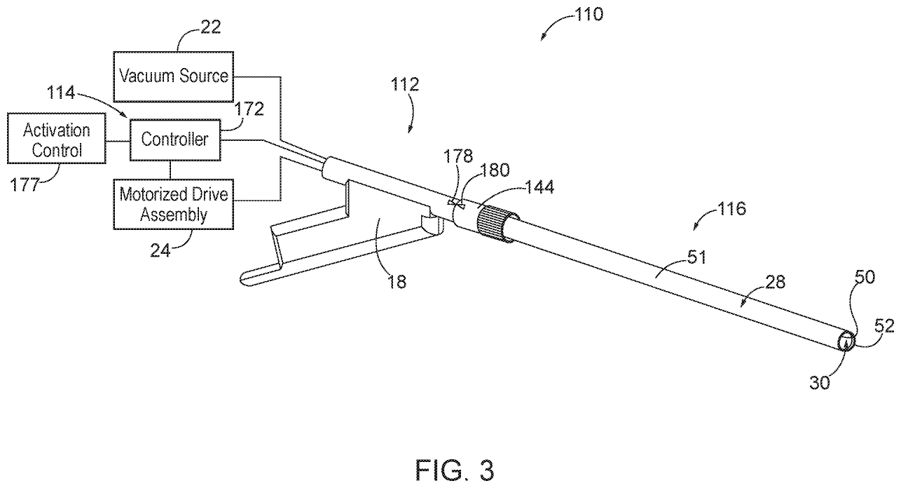

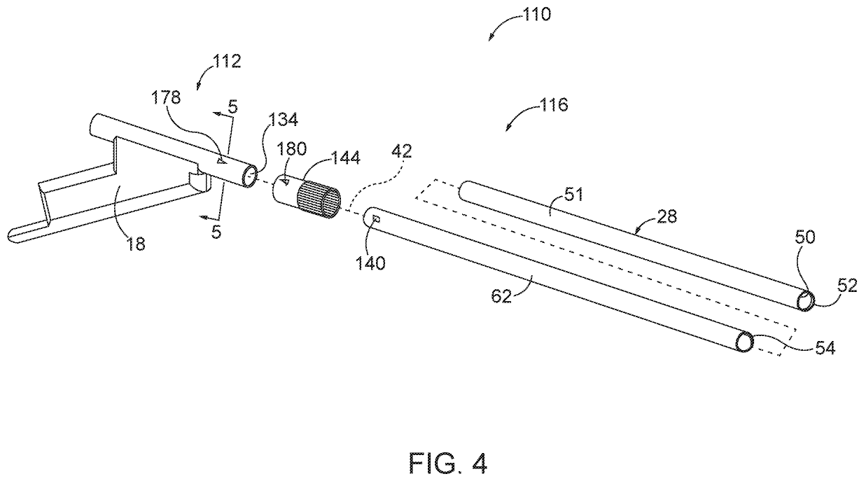

FIGS. 3-4 show a second exemplary surgical cutting instrument (110) including a handle assembly (112) and a shaft assembly alignment system (114) operatively connected to a second shaft assembly (116) for cutting and removing tissue as discussed above in greater detail. Handle assembly (112) has a handle body (118) that may include controls for the operation of surgical cutting instrument (10), or the controls may be located remotely. One such control is an activation control (not shown) configured to selectively power motorized drive assembly (24) via power source (26) (see FIG. 1). Vacuum source (22) fluidly connects to suction port (20) (see FIG. 1) to draw a vacuum through suction lumen (60) of cutting member (30) rotatably disposed within outer shaft (28). Motorized drive assembly (24) rotatably drives cutting member (30) within outer shaft (28) such that cutting member (30) rotates cyclically and repeatedly through an open state and a closed state in relation to outer shaft (28). In the open state, cutting window opening (54) aligns with shaft window opening (50) to fluidly communicate the vacuum throughout to the environment for receiving and suctioning tissue therein. In contrast, in the closed state, tubular sidewall (62) of cutting member (30) aligns with and covers shaft window opening (50) to inhibit, and even terminate in some examples, further suctioning. Shaft assembly alignment system (114) is configured to urge movement of cutting member (30) from the open state toward the closed state such that cutting member (30) stops in the closed state to inhibit further suctioning of the tissue through shaft window opening (50).

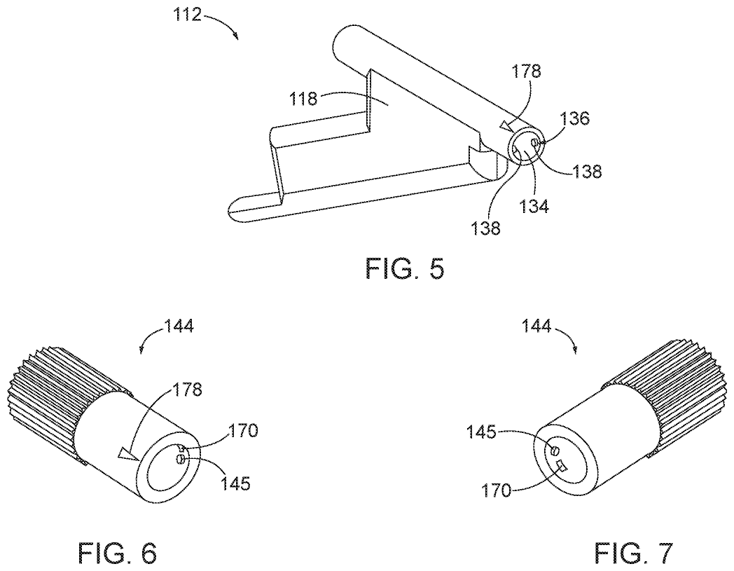

With respect to FIGS. 4-5, shaft assembly (116) extends distally from handle assembly (112) and connects to handle assembly (112) such that each of outer shaft (28) and cutting member (30) are configured to rotate relative to handle body (118). More particularly, cutting member (30) is rotatably received within a body lumen (134) in fluid communication with suction port (20) (see FIG. 1). A motor coupling (136) operatively connected to motorized drive assembly (24) is also positioned within body lumen (134) and configured to mechanically connect cutting member to motorized drive assembly (24) and transmit rotation therethrough. In the present example, motor coupling (136) has a pair of motor post mounts (138) respectively received within a pair of post holes (140) in a proximal end portion of cutting member (30) to affix the angular position of cutting member (30) relative to motor coupling (136).

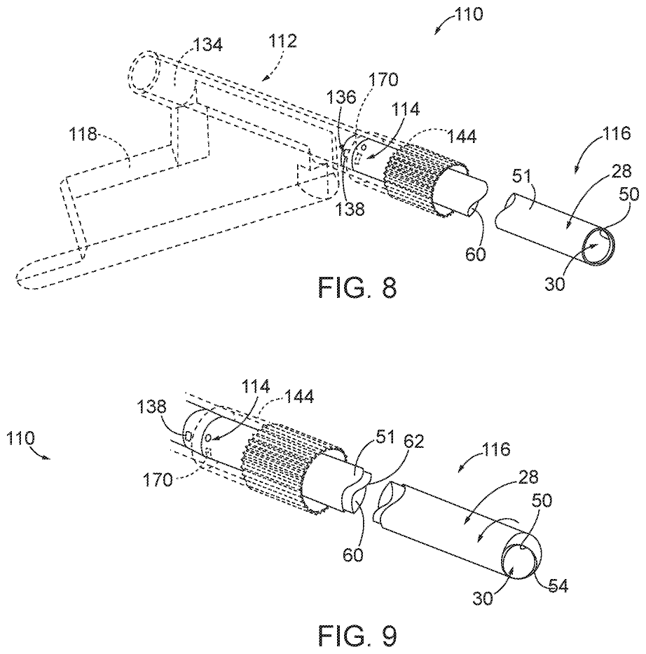

In addition, an outer coupling knob (144) of shaft assembly (116) rotatably connects outer shaft (28) to a distal end of handle body (118) as shown in FIGS. 6-9. Outer coupling knob (144) rigidly connects to outer shaft (28) via a pair of coupling posts (145) to affix the angular position of outer shaft to outer coupling knob (144), whereas outer coupling knob (144) is configured to rotate relative to handle body (118). Thereby, an operator may selectively rotate outer shaft (28) via outer coupling knob (144) to angularly position shaft window opening (50) to a desired angular position for more conveniently accessing target tissue for removal. Each of outer shaft (28) and cutting member (30) is thus independently rotatable relative to each other and to handle body (118).

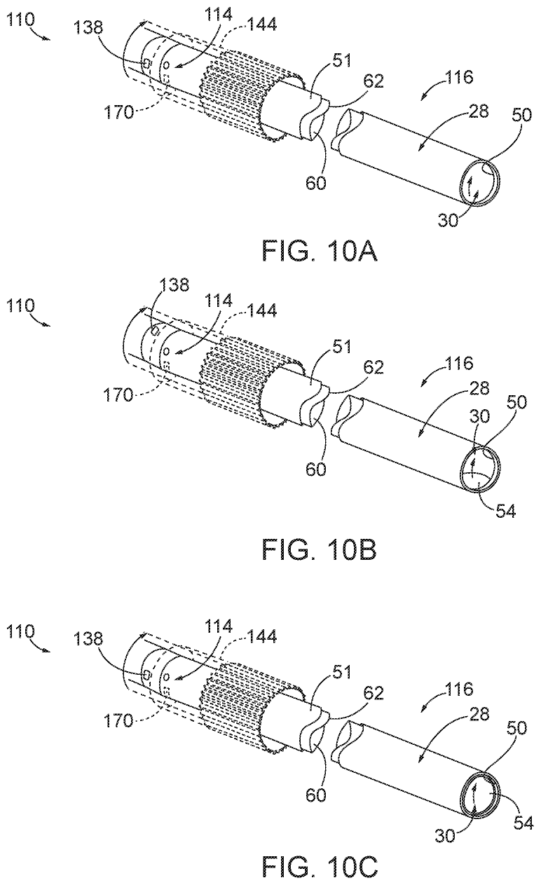

As discussed briefly above, shaft assembly alignment system (114) is configured to stop cutting member (30) relative to outer shaft (28) in the closed state to terminate suction through shaft window opening (50) to inhibit inadvertently damaging a desirable tissue. In the present example, shaft assembly alignment system (114) includes a pair of sensors, such as a pair of search coil sensors (170), configured to detect the angular position of cutting member (30) relative to outer shaft (28) for determining when the cutting member is in the closed state. Search coil sensors (170) are positioned within outer coupling knob (144) as shown more particularly in FIGS. 6-7 and configured to detect the position of respective motor post mounts (138) shown in FIGS. 10A-10C.

To this end, cutting window opening (54) is in the closed state relative to shaft window opening (50) in FIG. 10A such that search coil sensors (170) are configured to detect the proximally aligned motor post mounts (138). Of course, the angular position of cutting window opening (54) is fixed relative to the motor post mounts (138), and the angular position of shaft window opening (50) is fixed relative to the search coil sensors (170). These fixed angular positions in conjunction with detecting the variable position of cutting member (30) relative to outer shaft (28) collectively provide sufficient positional information to determine the alignment of cutting window opening (54) relative to shaft window opening (50). Rotating cutting window opening (54) from the closed state to the open state as shown in FIGS. 10B-10C misaligns search coil sensors (170) from motor post mounts (138) such that motor post mounts (138) are undetected. In the present example, the closed state occurs when tubular sidewall (62) completely covers shaft window opening (50) to terminate suction therethrough. The open state occurs when any portion of cutting window opening (54) aligns with shaft window opening (50) to communicate suction therethrough. In this respect, such open and closed states may each include a variety of positions for cutting member (30) and outer shaft (28), and the invention is not intended to be unnecessarily limited to such states being single, discrete positions.

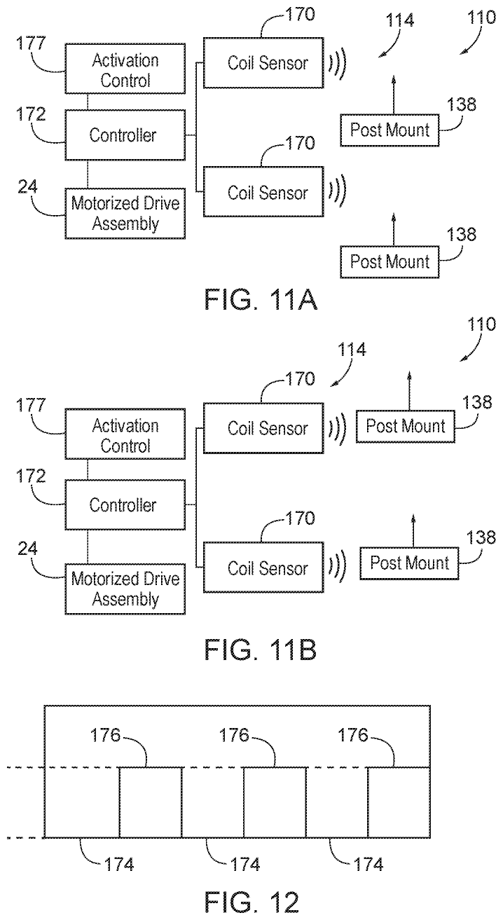

In use, FIGS. 11A-12 diagrammatically show shaft assembly alignment system (114) having a controller (172) connected to search coil sensors (170) and motorized drive assembly (24) to determine when the cutting window opening (54) is in the closed state and direct the motorized drive assembly (24) to urge movement of cutting member (30) as discussed above with respect to FIG. 10A. With reach cyclical rotation of cutting member (30), search coil sensors (170) detect the position of motor post mounts (138) and communicate such positions to controller (172) as an undetected signal (174) and a detected signal (176). Controller (172) allows motorized drive assembly (24) to continue rotating cutting member (30) so long as the operator selectively manipulates an activation control (177) to power motorized drive assembly (24).

After the operator manipulates activation control (177) to selectively cease powering motorized drive assembly (24), controller (172) directs motorized drive assembly (24) to urge cutting member (30) from the open state to the closed state based on the detected position of motor post mounts (138). In one example, such urging by motorized drive assembly (24) is more particularly arresting movement of cutting member (30) to stop cutting member (30) in the closed state. In another example, such urging by motorized drive assembly (24) is more particularly powered driven movement of cutting member (30) to stop cutting member (30) in the closed state. Of course, any combination of arrested and driven movement of cutting member (30) by motorized drive assembly (24) may be directed by controller (172) such that the invention is not intended to be unnecessarily limited to only arresting or driven movement of cutting member (30).

As discussed above, search coil sensors (170) are positioned on outer coupling knob (144) in order to directly detect the position of cutting member (30). In an alternative example, one or more search coil sensors (170) are positioned on handle body (118) to detect the position of cutting member (30). However, detection of cutting member (30) relative to handle body (118) fails to provide the angular position of outer shaft (28) relative to handle body (118) in order to determine the position of cutting window opening (54) relative to shaft window opening (50). Thus, in one example as shown with respect to FIGS. 3-6, outer shaft (28) may be manipulated by the operator to a predetermined shaft angular position before or after stopping movement of cutting member (30) in a predetermined cutting member angular position. More particularly, controller (172) may be configured to stop cutting member (30) based on the detected motor post mounts (138) in the predetermined cutting member angular position. Manipulating outer shaft (28) to the predetermined shaft angular position effectively aligns shaft assembly (116) in the closed state. A handle body indicator (178) on handle body (118) and an outer shaft indicator (180) on outer coupling knob (144) may be configured to cooperatively indicate the predetermined shaft alignment to the operator. It will be appreciated that alternative structures and methods for such cooperative indication of predetermined positions to the operator may be similarly used, and the invention is not intended to be unnecessarily limited to the particular indicators (178, 180) shown and described herein.

B. Shaft Assembly Alignment System with Shaft Encoders

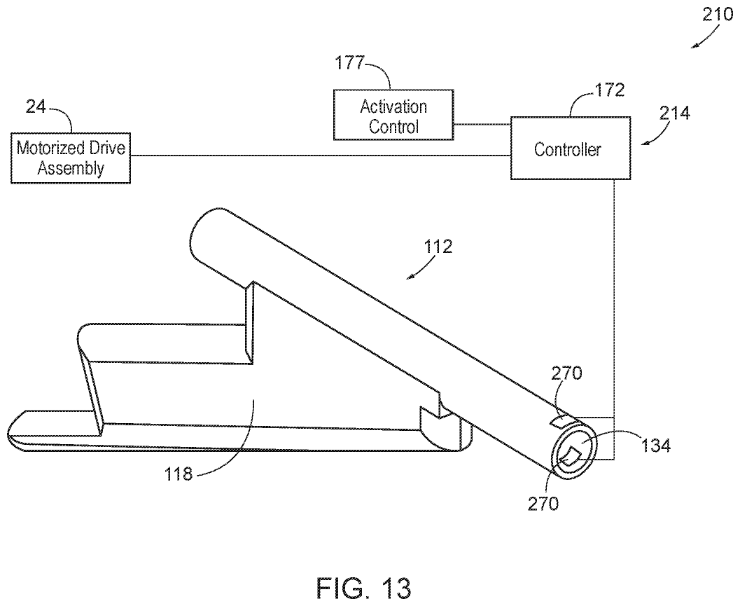

FIG. 13 shows a third exemplary surgical cutting instrument (210) having handle assembly (112), motorized drive assembly (24), and an alternative shaft assembly alignment system (214) configured to urge movement of cutting member (30) (see FIG. 10A) from the open state toward the closed state to inhibit suctioning the tissue through shaft window opening (50) (see FIG. 10A). Shaft assembly alignment system (214) has controller (172) operatively connected to a pair of sensors, such as a pair of shaft encoders (270), for respectively detecting angular positions of cutting member (30) (see FIG. 10A) and outer shaft (28) (see FIG. 10A). In this respect, controller (172) receives the detected positions for cutting member (30) and outer shaft (28) similar to search coil sensors (170) (see FIG. 11A) to determine the open and closed states and stop movement of cutting member (30) (see FIG. 10A) as discussed above. However, each shaft encoder (270) is positioned on handle body (118) respectively adjacent to cutting member (30) (see FIG. 10A) and outer shaft (28) (see FIG. 10A) to detect the angular positions of cutting member (30) (see FIG. 10A) relative to handle body (118) and outer shaft (28) (see FIG. 10A) relative to handle body (118). Controller (172) in the present example is thus configured to determine the angular positions of outer shaft (28) (see FIG. 10A) relative to cutting member (30) (see FIG. 10A) based on the detected angular positions communicated by shaft encoders (270). Alternatively, or in addition, shaft encoder (270) may be positioned between cutting member (30) (see FIG. 10A) and outer shaft (28) (see FIG. 10A) for similar detection. It will be appreciated that, in use, surgical cutting instrument (210) with shaft assembly alignment system (214) operates similar to surgical cutting instrument (110) discussed above with respect to FIGS. 10A-12.

C. Suction Alignment System with Pressure Sensor

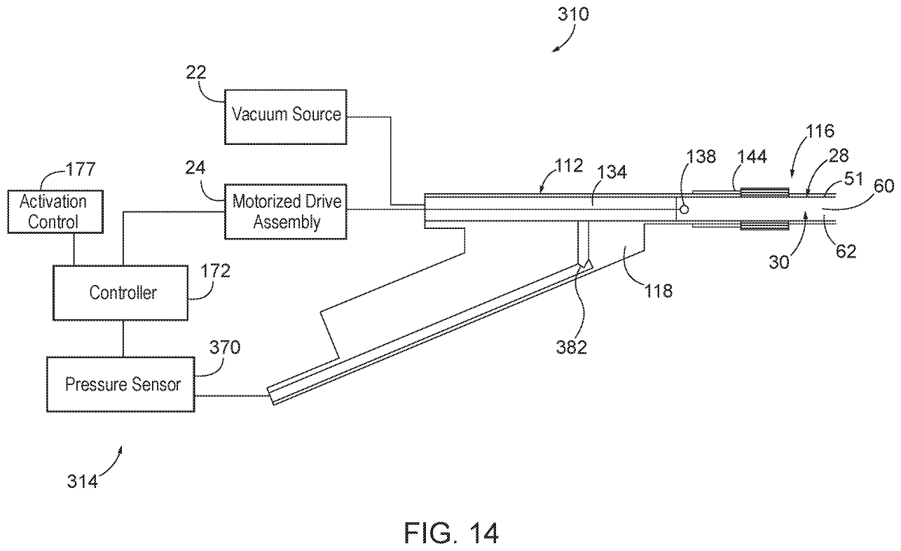

FIG. 14 shows a fourth exemplary surgical cutting instrument (310) having handle assembly (112), motorized drive assembly (24), and a suction alignment system (314) configured to urge movement of cutting member (30) from the open state toward the closed state to inhibit suctioning the tissue through shaft window opening (50) (see FIG. 10A). Suction alignment system (314) has controller (172) operatively connected to a sensor, such as a pressure sensor (370) for detecting the open and closed states of cutting member (30) and outer shaft (28) and stopping cutting member (30). In contrast to shaft assembly alignment system (114, 214), which detect one or more angular positions of cutting member (30) or outer shaft (28), pressure sensor (370) detects the loss and accumulation of the vacuum within suction lumen (60). Based on this loss or accumulation of vacuum, controller (172) determines whether cutting member (30) is in the open or closed state.

To this end, handle body (118) of the present example further includes a pressure conduit (382) fluidly connected to body lumen (134). Pressure sensor (370) fluidly connects to pressure conduit (382) to measure the detected pressure generated in suction lumen (60) via vacuum source (22) as cutting member (30) cycles through open and closed states. In the closed state, pressure in suction lumen (60) decreases to a predetermined low pressure, whereas pressure in suction lumen (60) increases to a predetermined high pressure. For example, the predetermined low pressure may be as low as the maximum vacuum generated by vacuum source (22), while the predetermined high pressure may be as high as the atmospheric pressure in the surrounding environment. The terms “high” and “low” with respect to pressure are thus merely relative terms and not intended to indicate a particular positive or negative pressure accumulation.

Once receiving the detected pressure from pressure sensor (370), controller (172) correlates the predetermined low pressure to the closed state and the predetermined high pressure to the open state. Additionally, pressure measurements between the predetermined low pressure and the predetermined high pressure may also be correlated to open and closed states as desired for greater accuracy in identifying the closed state from the open state. In other respects, in use, surgical cutting instrument (310) with shaft assembly alignment system (314) operates similar to surgical cutting instrument (110) discussed above with respect to FIGS. 10A-12.

D. Engagement Alignment System with Frictional Detent

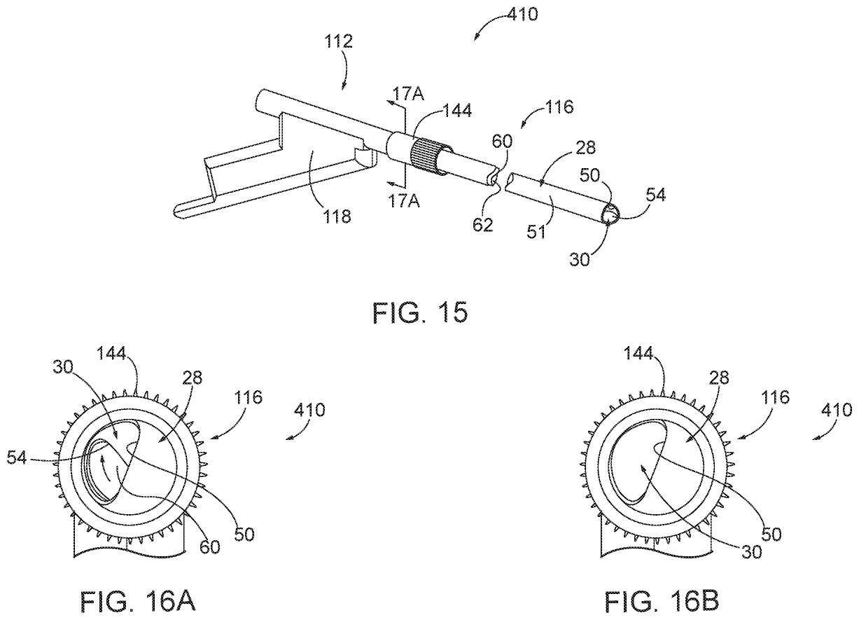

FIGS. 15-17B show a fifth exemplary surgical cutting instrument (410) having handle assembly (112) with handle body (118) and shaft assembly (116) with outer shaft (28). Surgical cutting instrument (410) further includes an engagement alignment system (414) configured to urge movement of cutting member (30) from the open state toward the closed state to inhibit suctioning the tissue through shaft window opening (50). More particularly, engagement alignment system (414) includes a detent (484) between cutting member (30) and outer shaft (28) configured to generate frictional engagement therebetween when cutting member (30) is in the closed state to encourage cutting member (30) to stop in the closed state.

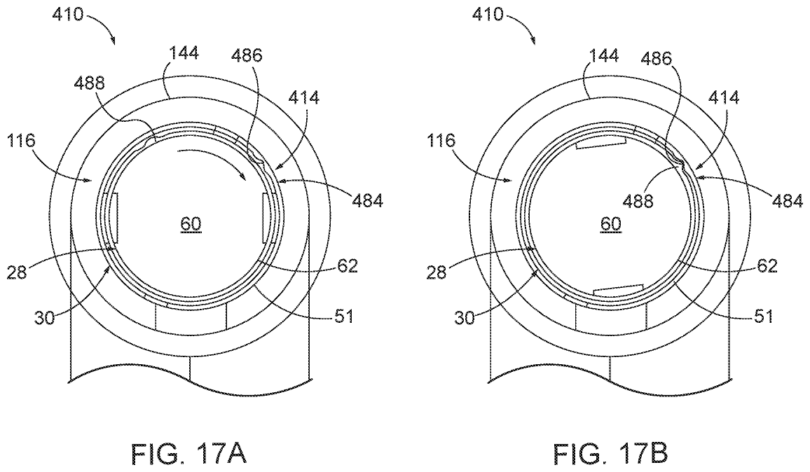

In the present example shown in FIGS. 17A-17B, detent (484) includes an outer abutment (486) extending radially inward from outer shaft (28) toward cutting member (30) and an inner abutment (488) extending radially outward from cutting member (30) toward outer shaft (28). Outer and inner abutments (486, 488) freely rotate by respective cutting member (30) and outer shaft (28), but radially overlap with each other to generate the frictional engagement as cutting member (30) is rotatably driven by motorized drive assembly (24) (see FIG. 3). Thereby, frictional engagement is relatively small so as to efficiently rotate cutting member (30) while cutting tissue, but sufficiently high to arrest cutting member (30) to stop in the closed state. While outer and inner abutments (486, 488) may be positioned in a variety of angular positions about shaft assembly (116), each of outer and inner abutments (486, 488) are positioned angularly opposite from cutting window opening (54) in the present example.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) a shaft extending along a longitudinal axis and including a shaft opening in fluid communication with an environment; (b) a cutting member disposed within the shaft and configured to cyclically move relative to the shaft from an open state to a closed state, wherein the cutting member is configured to cut a tissue and includes: (i) a sidewall, (ii) a cutting window opening extending through the sidewall, and (iii) a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough; and (c) an alignment system configured to urge movement of the cutting member from the open state toward the closed state such that the cutting member stops in the closed state to inhibit further suctioning of the tissue through shaft opening.

### Example 2

The surgical instrument of Example 1, wherein the alignment system includes a first sensor configured to detect when the cutting member is in the closed state.

### Example 3

The surgical instrument of Example 2, wherein the first sensor is positioned on the shaft.

### Example 4

The surgical instrument of any one or more of Examples 2 through 3, further comprising a motor coupling configured to mechanically connect the cutting member to a motorized drive assembly, wherein an angular position of the motor coupling mechanically connected to cutting member is fixed, and wherein the first sensor is configured to detect at least a portion of the motor coupling when the cutting member is in the closed state.

### Example 5

The surgical instrument of any one or more of Examples 2 through 4, wherein the alignment system includes a second sensor configured to detect when the cutting member is in the closed state.

### Example 6

The surgical instrument of any one or more of Examples 2 through 5, wherein the first sensor is a search coil sensor.

### Example 7

The surgical instrument of Example 2, further comprising a body having the shaft and the cutting member extending distally therefrom, wherein the first sensor is positioned on the body.

### Example 8

The surgical instrument of Example 7, wherein the body is configured to receive the cutting member rotatably thereagainst, and wherein the first sensor is configured detect a shaft angular position of the shaft relative to the body.

### Example 9

The surgical instrument of any one or more of Examples 7 through 8, wherein the first sensor is a first shaft encoder.

### Example 10

The surgical instrument of any one or more of Examples 7 through 9, wherein the body is further configured to receive the shaft rotatably thereagainst, wherein the alignment system further includes a second sensor positioned on the body, and wherein the second sensor is configured to detect a cutting member angular position of the cutting member relative to the body.

### Example 11

The surgical instrument of Example 10, wherein the first sensor is a first shaft encoder, and wherein the second sensor is a second shaft encoder.

### Example 12

The surgical instrument of Example 2, wherein the first sensor is a pressure sensor in fluid communication with the suction lumen and configured to detect a vacuum in the suction lumen when the cutting member is in the closed state.

### Example 13

The surgical instrument of Example 12, further comprising a body having a pressure conduit in fluid communication with the suction lumen and the shaft and the cutting member extending distally therefrom, and wherein the pressure sensor is fluidly connected to the pressure conduit.

### Example 14

The surgical instrument of any one or more of Examples 2 through 13, wherein the alignment system further includes a controller operatively connected to the first sensor and the cutting member, wherein the controller is configured to determine that the cutting member is in the closed state based on detection of the closed state by the first sensor and stop movement of the cutting member in the closed state.

### Example 15

The surgical instrument of any one or more of Examples 1 through 14, wherein the alignment system includes a detent positioned between the shaft and the cutting member, wherein the detent is configured to urge movement of the cutting member from the open state toward the closed state such that the cutting member stops in the closed state.

### Example 16

A surgical instrument, comprising: (a) a shaft extending along a longitudinal axis and including a shaft opening in fluid communication with an environment; (b) a cutting member disposed within the shaft and configured to cyclically move relative to the shaft from an open state to a closed state, wherein the cutting member is configured to cut a tissue and includes: (i) a sidewall, (ii) a cutting window opening extending through the sidewall, and (iii) a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough; (c) a motorized drive assembly connected to the cutting member and configured to drive the cutting member to cyclically move relative to the shaft; and (d) an alignment system including: (i) a sensor configured to detect when the cutting member is in the closed state, (ii) a controller operatively connected to the sensor and the motorized drive assembly, wherein the controller is configured to determine that the cutting member is in the closed state based on detection of the closed state by the sensor, wherein the controller is further configured to direct the motorized drive assembly to urge movement of the cutting member from the open state toward the closed state such that the cutting member stops in the closed state to inhibit further suctioning of the tissue through shaft opening.

### Example 17

The surgical instrument of Example 16, further comprising a motor coupling mechanically connecting the cutting member to the motorized drive assembly, wherein an angular position of the motor coupling mechanically connected to cutting member is fixed, and wherein sensor is configured to detect at least a portion of the motor coupling when the cutting member is in the closed state.

### Example 18

The surgical instrument of Example 16, further comprising a body having the shaft and the cutting member extending distally therefrom, wherein the body is configured to receive the cutting member rotatably thereagainst, and wherein the sensor is configured detect a shaft angular position of the shaft relative to the body.

### Example 19

The surgical instrument of Example 16, wherein the sensor is a pressure sensor in fluid communication with the suction lumen and configured to detect a vacuum in the suction lumen when the cutting member is in the closed state.

### Example 20

A method of cutting a tissue with a surgical instrument, wherein the surgical instrument includes a shaft extending along a longitudinal axis with a shaft opening in fluid communication with an environment; a cutting member disposed within the shaft and configured to cyclically move relative to the shaft from an open state to a closed state, wherein the cutting member is configured to cut the tissue and includes: a sidewall, a cutting window opening extending through the sidewall, and a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough; and an alignment system configured to urge movement of the cutting member moving from the open state toward the closed state such that the cutting member stops in the closed state to inhibit further suctioning of the tissue through shaft opening, the method comprising: (a) cutting the tissue with the cutting member in the open state; (b) moving the cutting member from the open state toward the closed state; (c) detecting the closed state with the alignment system; and (d) urging movement of the cutting member based on the detected closed state thereby stopping the cutting member in the closed state.

IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) a shaft extending along a longitudinal axis and including a shaft opening in fluid communication with an environment;
(b) a cutting member disposed within the shaft and configured to cyclically move relative to the shaft from an open state to a closed state, wherein the cutting member is configured to cut a tissue and includes:
(i) a sidewall,
(ii) a cutting window opening extending through the sidewall, and
(iii) a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough; and
(c) an alignment system configured to urge movement of the cutting member from the open state toward the closed state such that the cutting member stops in the closed state to inhibit further suctioning of the tissue through shaft opening.

2. The surgical instrument of claim 1, wherein the alignment system includes a first sensor configured to detect when the cutting member is in the closed state.

3. The surgical instrument of claim 2, wherein the first sensor is positioned on the shaft.

4. The surgical instrument of claim 3, further comprising a motor coupling configured to mechanically connect the cutting member to a motorized drive assembly, wherein an angular position of the motor coupling mechanically connected to cutting member is fixed, and wherein the first sensor is configured to detect at least a portion of the motor coupling when the cutting member is in the closed state.

5. The surgical instrument of claim 4, wherein the alignment system includes a second sensor configured to detect when the cutting member is in the closed state.

6. The surgical instrument of claim 4, wherein the first sensor is a search coil sensor.

7. The surgical instrument of claim 2, further comprising a body having the shaft and the cutting member extending distally therefrom, wherein the first sensor is positioned on the body.

8. The surgical instrument of claim 7, wherein the body is configured to receive the cutting member rotatably thereagainst, and wherein the first sensor is configured detect a shaft angular position of the shaft relative to the body.

9. The surgical instrument of claim 8, wherein the first sensor is a first shaft encoder.

10. The surgical instrument of claim 8, wherein the body is further configured to receive the shaft rotatably thereagainst, wherein the alignment system further includes a second sensor positioned on the body, and wherein the second sensor is configured to detect a cutting member angular position of the cutting member relative to the body.

11. The surgical instrument of claim 10, wherein the first sensor is a first shaft encoder, and wherein the second sensor is a second shaft encoder.

12. The surgical instrument of claim 2, wherein the first sensor is a pressure sensor in fluid communication with the suction lumen and configured to detect a vacuum in the suction lumen when the cutting member is in the closed state.

13. The surgical instrument of claim 12, further comprising a body having a pressure conduit in fluid communication with the suction lumen and the shaft and the cutting member extending distally therefrom, and wherein the pressure sensor is fluidly connected to the pressure conduit.

14. The surgical instrument of claim 2, wherein the alignment system further includes a controller operatively connected to the first sensor and the cutting member, wherein the controller is configured to determine that the cutting member is in the closed state based on detection of the closed state by the first sensor and stop movement of the cutting member in the closed state.

15. The surgical instrument of claim 1, wherein the alignment system includes a detent positioned between the shaft and the cutting member, wherein the detent is configured to urge movement of the cutting member from the open state toward the closed state such that the cutting member stops in the closed state.

16. A surgical instrument, comprising:
(a) a shaft extending along a longitudinal axis and including a shaft opening in fluid communication with an environment;
(b) a cutting member disposed within the shaft and configured to cyclically move relative to the shaft from an open state to a closed state, wherein the cutting member is configured to cut a tissue and includes:
(i) a sidewall,
(ii) a cutting window opening extending through the sidewall, and
(iii) a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough;
(c) a motorized drive assembly connected to the cutting member and configured to drive the cutting member to cyclically move relative to the shaft; and
(d) an alignment system including:
(i) a sensor configured to detect when the cutting member is in the closed state,
(ii) a controller operatively connected to the sensor and the motorized drive assembly, wherein the controller is configured to determine that the cutting member is in the closed state based on detection of the closed state by the sensor,
wherein the controller is further configured to direct the motorized drive assembly to urge movement of the cutting member from the open state toward the closed state such that the cutting member stops in the closed state to inhibit further suctioning of the tissue through shaft opening.

17. The surgical instrument of claim 16, further comprising a motor coupling mechanically connecting the cutting member to the motorized drive assembly, wherein an angular position of the motor coupling mechanically connected to cutting member is fixed, and wherein sensor is configured to detect at least a portion of the motor coupling when the cutting member is in the closed state.

18. The surgical instrument of claim 16, further comprising a body having the shaft and the cutting member extending distally therefrom, wherein the body is configured to receive the cutting member rotatably thereagainst, and wherein the sensor is configured detect a shaft angular position of the shaft relative to the body.

19. The surgical instrument of claim 16, wherein the sensor is a pressure sensor in fluid communication with the suction lumen and configured to detect a vacuum in the suction lumen when the cutting member is in the closed state.

20. A method of cutting a tissue with a surgical instrument, wherein the surgical instrument includes a shaft extending along a longitudinal axis with a shaft opening in fluid communication with an environment; a cutting member disposed within the shaft and configured to cyclically move relative to the shaft from an open state to a closed state, wherein the cutting member is configured to cut the tissue and includes: a sidewall, a cutting window opening extending through the sidewall, and a suction lumen extending along the longitudinal axis and in fluid communication with the cutting window opening, wherein the cutting window opening in the open state aligns with the shaft opening such that the cutting window opening and the shaft opening are in fluid communication for suctioning the tissue therethrough, wherein the sidewall in the closed state aligns with the shaft opening such that the sidewall blocks fluid communication to the shaft opening for inhibiting suctioning the tissue therethrough; and an alignment system configured to urge movement of the cutting member moving from the open state toward the closed state such that the cutting member stops in the closed state to inhibit further suctioning of the tissue through shaft opening, the method comprising:
(a) cutting the tissue with the cutting member in the open state;
(b) moving the cutting member from the open state toward the closed state;
(c) detecting the closed state with the alignment system; and
(d) urging movement of the cutting member based on the detected closed state thereby stopping the cutting member in the closed state.
